(19) United States
(12) Patent Application Publication
(10) Pub. No.: US 2014/0303427 A1
(43) Pub. Date: Oct. 9, 2014

(54) BLOOD FLOW SYSTEM WITH OPERATOR ATTACHABLE COMPONENTS

(71) Applicant: CIRCULITE, INC., Teaneck, NJ (US)

(72) Inventors: Wolfgang Kerkhoffs, Aachen (DE); Ellen Keysselitz, Aachen (DE); Oliver Marseille, Aachen (DE)

(73) Assignee: CIRCULITE, INC., Teaneck, NJ (US)

(21) Appl. No.: 14/247,655

(22) Filed: Apr. 8, 2014

Related U.S. Application Data
(60) Provisional application No. 61/809,984, filed on Apr. 9, 2013.

(51) Int. Cl. A61M 1/10; A61M 1/12

(52) U.S. Cl. CPC A61M 1/1008; A61M 1/1029; A61M 1/125
USPC 600/16

(57) ABSTRACT

A system for pumping blood in a patient comprises multiple components including a rotational drive assembly; a fluid drive element; a housing, an inflow cannula assembly and an outflow graft assembly. The inflow cannula assembly comprises an inflow conduit and an inflow connector. The outlet graft assembly comprises an outflow conduit and an outflow connector. The housing surrounds the fluid drive element and comprises an inflow port and an outflow port. The inflow conduit comprises a first end operably attachable to the inflow port and a second end fluidly connectable to a source of oxygenated blood. The outflow conduit comprises a first end operably attachable to the outflow port and a second end fluidly connectable to a blood vessel. The inflow connector operably connects the inflow conduit to the inflow port. The outflow connector operably connects the outflow conduit to the outflow port.

### CROSS-REFERENCE TO RELATED APPLICATIONS

This application claims the benefit of U.S. Provisional Application Ser. No. 61/809,984 filed Apr. 9, 2013 (pending). This application is also related to U.S. patent application Ser. No. 09/202,538, entitled “Blood Pump”, filed Dec. 16, 1998 (now U.S. Pat. No. 6,116,862); U.S. patent application Ser. No. 09/155,818, entitled “Intravascular Blood Pump”, filed Oct. 5, 1998 (now U.S. Pat. No. 6,176,848); U.S. patent application Ser. No. 12/392,623, entitled “Devices, Methods and Systems for Establishing Supplemental Blood Flow in the Circulatory System”, filed Feb. 25, 2009; U.S. patent application Ser. No. 12/872,394, entitled “Two Piece Endovascular Anastomotic Connector”, filed Aug. 31, 2010 (now U.S. Pat. No. 8,333,727), the disclosures of which are each incorporated herein by reference in their entirety.

### FIELD OF THE INVENTION

The present invention relates generally to medical devices, systems and methods, and more particularly, to device and methods for assisting in the conduction of bodily fluids such as blood.

### BACKGROUND OF THE INVENTION

Various devices, systems and methods have been utilized to assist in conducting bodily fluids. For instance, blood pumps with inflow and outflow graft assist the heart in circulating blood in a patient experiencing congestive heart failure, and a transplant organ has either not been located or the patient is not a suitable candidate for the transplant. Accordingly, the blood pump can be fluidically attached to the left side of the heart and then located remotely, such as subcutaneously or submuscularly in a manner similar to a pacemaker, in what is referred to as a “pump pocket.” The pump pocket can be generally located at a position that is accessible by a surgical incision from below the collarbone, over the pectoral muscle, and toward the breast. A cannula can then be used to fluidically couple the heart to the pump. In still another example, a cannula is inserted into the bladder or kidney, such as in dialysis or to treat urinary obstruction or infection.

A fluid drive device, such as a pump, can be used to circulate the bodily fluid. Areas of insufficient flow, such as low-flow areas within or proximate to the fluid drive device, can result in the circulated fluid undesirably transitioning to solid matter. With blood pumping systems, blood in a stasis or near-stasis condition can transition to thrombus. Creation of thrombus or other solid matter can result in reduced flow of the fluid drive device or, more significantly, release of solid matter into the patient such as a released embolus that causes a stroke, heart attack, or other ischemic event. Blood pump implantation procedures include making precise measurements to properly size (e.g. cur to length) flow conduits and require specific order of flow conduit attachments (e.g. order of attachment to body lumens).

For these and other reasons, there is a need for devices, systems and methods which reduce the likelihood of inadequate flow conditions and creation of emboli, while providing flexibility and security of flow conduit attachment and other surgical implantation steps. Desirably, the systems, methods and devices will improve long term efficacy and minimize device complications.

### SUMMARY

According to an aspect of the invention, a system for pumping blood in a patient comprises a rotational drive assembly; a fluid drive element; a housing surrounding the fluid drive element and comprising an inflow port and an outflow port; an inflow cannula assembly; and an outflow graft assembly. The inflow cannula assembly comprises a proximal end and a distal end, where the distal end is operably attachable to the inflow port and the proximal end is fluidly connectable to a source of oxygenated blood, and an inflow connector constructed and arranged to operably connect the inflow conduit to the inflow port. The outflow graft assembly comprises an outflow conduit with a proximal end and a distal end, where the proximal end is operably attachable to the outflow port and the distal end is fluidly connectable to a blood vessel, and an outflow connector constructed and arranged to operably connect the outflow conduit to the outflow port.

The system can be constructed and arranged to allow an operator to attach the inflow conduit to the inflow port prior to or after attaching the outflow conduit to the outflow port.

The system can be constructed and arranged to allow an operator to anastomose the outflow conduit to a blood vessel prior to attaching the outflow conduit to the outflow port.

The system can be constructed and arranged to allow an operator to prime the system from the outflow port to the inflow port.

The system can further comprise a flow pathway from the inflow conduit proximal end to the outflow conduit distal end. The flow pathway can be constructed and arranged to minimize at least one of: turbulence; flow stagnation; and a low-flow location.

The system can be constructed and arranged to allow priming by causing blood to flow from the outflow port to the inflow port.

The system can be constructed and arranged to allow priming with blood without providing power to the rotational drive assembly.

The inflow cannula assembly can be constructed and arranged to provide feedback of proper attachment. For example, the feedback can comprise feedback selected from the group consisting of: audible feedback; tactile feedback; force feedback; and combinations thereof.

The inflow conduit can comprise a material selected from the group consisting of: silicone; polyurethane, polyester, expanded polytetrafluoroethylene (ePTFE); and combinations thereof.

The inflow conduit can comprise a multi-layer construction.

The inflow conduit can further comprise a flange proximate the inflow conduit distal end.

The inflow conduit can comprise a first length constructed and arranged to be reduced by an operator.

The inflow conduit can comprise a first segment proximate the distal end and a second segment continuous with the first segment, where the second segment comprises a wall thickness, and the first segment comprises a wall thickness thicker than the second segment wall thickness.

The inflow conduit can comprise a first segment proximate the distal end and a second segment continuous with the first segment, where the second segment comprises a reinforcing element along its length and the first segment does not include a reinforcing element. The inflow connector can be constructed and arranged to be positioned on the inflow conduit first segment.

The inflow conduit can be constructed and arranged to be fluidly connected to a left atrium.

The inflow conduit can comprise a lumen with a diameter between approximately 2 mm and 10 mm.

The inflow connector can be constructed and arranged to provide feedback when sufficient connection between the inflow conduit and the inflow port is achieved. For example, the feedback can be selected from the group consisting of: tactile feedback; audible feedback; and combinations thereof.

The inflow connector can be constructed and arranged to rotatably engage the inflow port.

The inflow connector can comprise at least one radially inward facing projection. The inlet port can comprise a recess constructed and arranged to slidingly receive the at least one projection. The recess can comprise a retention portion constructed and arranged to engage the at least one projection and prevent rotation of the inlet connector.

The inflow connector can comprise a bayonet locking element.

The outflow graft assembly can be constructed and arranged to provide feedback of proper attachment. For example, the feedback can be selected from the group consisting of: tactile feedback; audible feedback; and combinations thereof.

The outflow conduit can comprise a material selected from the group consisting of: ePTFE; a polyester weave such as a sealed woven polyester material; and combinations thereof.

The outflow conduit can comprise a multi-layer construction. For example, the outflow conduit can comprise an inner layer, an outer layer and a hydrostatic barrier middle layer.

The outflow conduit can comprise a lumen with a diameter between approximately 4 mm and 12 mm.

The outflow conduit can comprise a first length constructed and arranged to be reduced by an operator.

The outflow conduit can be constructed and arranged to be fluidly attached to a subclavian artery.

The outflow connector can be constructed and arranged to rotatably engage the outflow port.

The outflow connector can be pre-attached to the outflow conduit.

The outflow connector can be constructed and arranged to be tightened to attach the outflow conduit to the outflow port, and the outflow connector can be further constructed and arranged to allow rotation of the outflow conduit at least one of during or after the tightening of the outflow connector. The outflow connector can be further constructed and arranged to allow low-torque rotation of the outflow conduit at least one of during or after the tightening of the outflow connector.

The inflow port can comprise an outer diameter, and the inflow conduit can be constructed and arranged to create a fluid seal with the inflow port outer diameter.

The outflow port can comprise an outer diameter, and the outflow conduit can be constructed and arranged to create a fluid seal with the outflow port outer diameter.

The system can further comprise a third port constructed and arranged to be operably attached to a cable. The system can further comprise a second cable positioned between the housing and the third port. The system can further comprise a fluid drive module constructed and arranged to rotate the fluid drive element, where the cable comprises one or more electrical wires constructed and arranged to provide electrical energy to the fluid drive module. The cable can comprise a first cable and a second cable attachable to the first cable, for example where the system further comprises an external power supply, and the first cable is attachable to the third port and the second cable is attachable to the external power supply.

The cable can comprise at least a first end and a second end, and the system can further comprise at least one protective cover for attachment to at least one of the first end or the second end. The protective cover can comprise a material selected from the group consisting of: foam; porous material; polyethylene; polyethylene foam; high-density polyethylene; ultra-high-molecular weight polyethylene; polytetrafluoroethylene; porous polytetrafluoroethylene; hydrophilic material; and combinations thereof.

The cable can comprise a rotatable shaft constructed and arranged to cause the fluid drive element to rotate. The cable can comprise a source of at least one of pneumatic or hydraulic fluid constructed and arranged to cause the fluid drive element to rotate.

The system can further comprise a tightening tool. The tightening tool can comprise a disengaging assembly constructed and arranged to prevent applying a torque above a threshold torque level. The disengaging assembly can comprise a locking element, a spring and a capture recess, and the disengaging assembly can be constructed and arranged such that the locking element is biased into the capture recess by the spring. The disengaging assembly can be constructed and arranged such that the locking element disengages from the capture recess when an applied torque reaches the threshold torque level. The tightening tool can be constructed and arranged to tighten the inflow connector and/or the outflow connector. The tightening tool can be constructed and arranged to prevent applying a torque to the inflow connector and/or outflow connector above a threshold torque level.

The system can further comprise a fluid flow clamp. The clamp can comprise a Kelly clamp. The clamp can be constructed and arranged to at least partially occlude flow in a component selected from the group consisting of: the inflow conduit; an artery of the patient; the outflow conduit; and combinations thereof. The system can further comprise any number of clamps, for example a second and a third clamp.

According to another aspect of the invention, a method of implanting a system for pumping blood in a patient comprises selecting the blood flow system in any configuration described above, and implanting the housing, the inflow cannula assembly and the outflow graft assembly in the patient. The outflow graft assembly can be attached to a blood vessel. For example, the blood vessel can comprise a subclavian artery. Subsequently, the outflow graft assembly can be attached to the outflow port. Subsequently, blood can be flowed through the housing in a retrograde direction. Subsequently, the inflow cannula assembly can be attached to the inlet port. The method can further comprise reducing a first length of the inflow conduit and/or the outflow conduit. In some embodiments, the method can be performed in a different order.

The technology described herein, along with the attributes and attendant advantages thereof, will best be appreciated and understood in view of the following detailed description taken in conjunction with the accompanying drawings in which representative embodiments are described by way of example.

### BRIEF DESCRIPTION OF THE DRAWINGS

FIG. 1 is a schematic view of a fluid flow system for a patient, including a fluid pump and operator attachable components, consistent with the present inventive concepts.

FIG. 2 is a perspective view of an inflow cannula assembly positioned for attachment to an inflow port of a fluid pump, consistent with the present inventive concepts.

FIG. 2A is a side view of the inflow port of the fluid pump of FIG. 2, consistent with the present inventive concepts.

FIG. 2B is a side sectional view of the inflow cannula assembly of FIG. 2 attached to the fluid pump inflow port of FIG. 2A, consistent with the present inventive concepts.

FIGS. 3A-3D are side sectional and perspective views of a group of attachment components of an outflow graft assembly including an independently rotatable collar, consistent with the present inventive concepts.

FIG. 3E is a side sectional and end views of an outflow port of a fluid pump, consistent with the present inventive concepts.

FIG. 4 is a side sectional view of an end of the outflow graft assembly comprising the components of FIGS. 3A-3D positioned for attachment to the fluid pump outflow port of FIG. 3E, consistent with the present inventive concepts.

FIG. 5 is a side sectional view of an implantable male electrical connector, consistent with the present inventive concepts.

FIG. 6 is a side sectional view of an implantable female electrical connector, consistent with the present inventive concepts.

FIG. 7A is a side view of a tool for attaching two components of a fluid flow system, consistent with the present inventive concepts.

FIG. 7B is a side view of the tool of FIG. 7A after a torque-limiting mechanism has been activated.

FIG. 8 is a side sectional view of a distal end of an inflow cannula assembly attached to an inflow port of a fluid pump, consistent with the present inventive concepts.

FIG. 8A is a perspective view of the fluid pump inflow port of FIG. 8, consistent with the present inventive concepts.

FIG. 8B is a perspective view of the distal portion of the inflow cannula assembly of FIG. 8, consistent with the present inventive concepts.

FIGS. 9A and 9B are side sectional and perspective views, respectively, of a fluid absorbing plug, consistent with the present inventive concepts.

### DETAILED DESCRIPTION OF THE DRAWINGS

Reference will now be made in detail to the present embodiments of the technology, examples of which are illustrated in the accompanying drawings. The same reference numbers are used throughout the drawings to refer to the same or like parts.

The terminology used herein is for the purpose of describing particular embodiments and is not intended to be limiting of the inventive concepts. As used herein, the singular forms “a,” “an” and “the” are intended to include the plural forms as well, unless the context clearly indicates otherwise.

It will be further understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be understood that, although the terms first, second, third etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element or intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

Spatially relative terms, such as “beneath,” “below,” “lower,” “above,” “upper” and the like may be used to describe an element and/or feature's relationship to another element(s) and/or feature(s) as, for example, illustrated in the figures. It will be understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g., rotated 90 degrees or at other orientations) and the spatially relative descriptors used herein interpreted accordingly.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

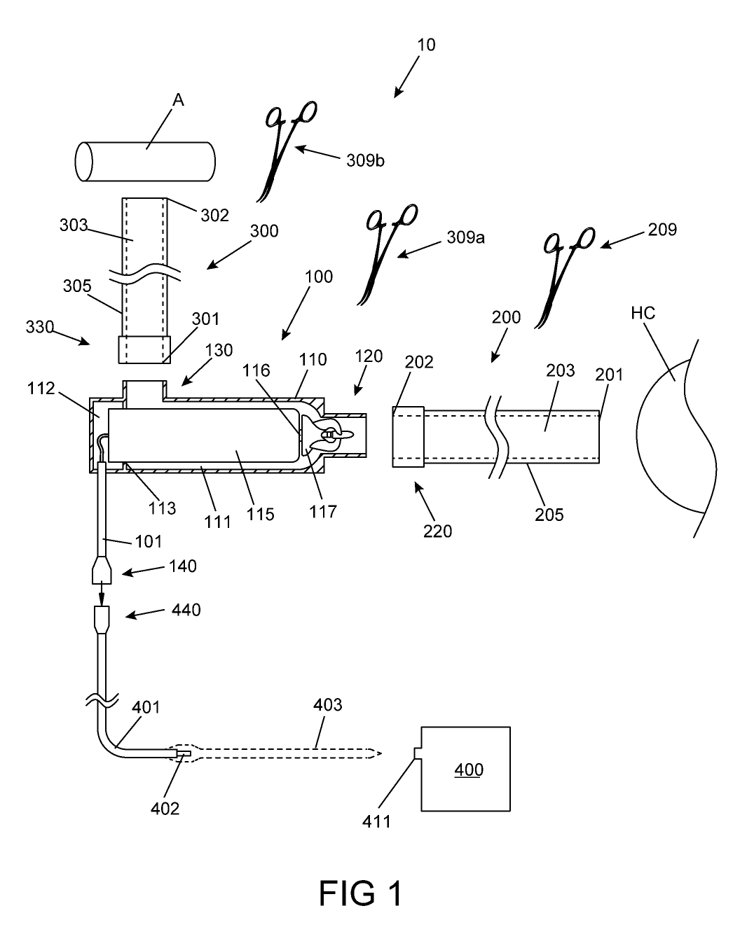

Referring now to FIG. 1, a schematic view of a fluid flow system for a patient is illustrated, including a fluid flow pump and operator attachable components, consistent with the present inventive concepts. System 10, typically a blood flow system, includes pump 100. System 10 further includes inflow cannula assembly 200, outflow graft assembly 300, and a power supply, such as control module 400. Pump 100 includes housing 110 which comprises one or more rigid or relatively rigid metal or plastic materials, such as stainless steel or titanium materials. Housing 110 is typically implanted in a subcutaneous pocket, under the skin of the patient. Housing 110 surrounds a rotational drive assembly that includes motor 115. Housing 110 further surrounds fluid drive element 117 which is operably attached to motor 115. In some embodiments, system 10 comprises a rotational drive assembly similar to that described in the above incorporated U.S. Pat. No. 6,116,862, or a rotational drive assembly similar to that described in the above incorporated U.S. Pat. No. 6,176,848. Fluid drive element 117, for example an impeller, is engaged to motor 115, for example an electromagnetic motor, by shaft 116 such that fluid drive element 117 can be rotated by motor 115. Fluid drive element 117 can be rotated by direct drive of shaft 116, or it can be rotated about shaft 116 by a magnetic coupling, such as a rotating magnetic field that engages one or more magnetic portions of drive element 117. In an alternative embodiment, fluid drive element 117 is magnetically levitated, avoiding the need for shaft 116.

Housing 110 comprises two fluid communication ports, inflow port 120 and outflow port 130. Inflow port 120 and outflow port 130 are fluidly connected to each other through housing 110 via a space created within housing, chamber 111. Fluid drive element 117 generates fluid propulsion forces such that fluid is drawn into housing 110 via inflow port 120, and flows through chamber 111 to exit through outflow port 130. A sealing surface or element, seal 113, circumferentially surrounds a portion of motor 115 such as to prevent fluid from passing into space 112, such as to isolate one or more components within space 112 from contacting fluid and/or to prevent a low flow area within housing 110. In some embodiments, chamber 111 comprises a volume less than 100 mL, for example less than 50 mL. In some embodiments, chamber 111 comprises a volume less than 10 mL, for example less than 5 mL, such as less than 2.5 mL or less than 1.2 mL.

System 10 can include one or more operator attachable connections, such as are described in detail herebelow. Operator attachable connections can comprise connections configured to operably (e.g. fluidly, electrically, mechanically and/or otherwise functionally) connect two or more assemblies or other components of system 10, such as operable connections between pump 100 and inflow cannula assembly 200, outflow graft assembly 300 and/or control module 400. These connections can be made by an operator such as a surgeon during a sterile clinical procedure, in any order. Operator attachable connections can be made during an initial implant procedure and/or during an implant revision procedure, such as a sterile clinical procedure performed in an operating room to implant, replace or upgrade one or more components of system 10. In some embodiments, pump 100 can be replaced, such as due to an electrical or mechanical failure of pump 100 (e.g. pump stoppage due to thrombus generation). In these embodiments, one or more of the remaining components of system 10 (e.g. inflow cannula assembly 200, outflow graft assembly 300 and/or control module 400 and/or the wire bundles attached thereto), can be simply detached from pump 100 to be replaced and attached to a newly implanted pump, such as a pump comprising similar and/or dissimilar components and functionality as compared to the pump 100 illustrated on FIG. 1. System 10 is constructed and arranged such that one or more of these assemblies can be easily and rapidly replaced while leaving other assemblies in tact.

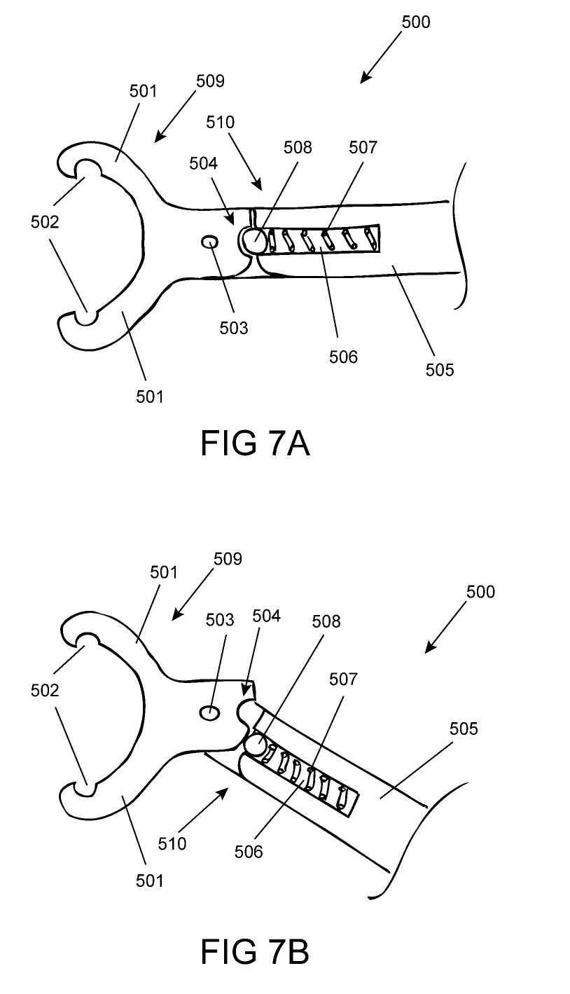

In some embodiments, one or more of the attachable components of system 10 are implanted in the patient (e.g. attached to a portion of the patient's cardiovascular system), prior to attachment to another component of system 10. Operator attachable connections can be configured to provide feedback (e.g. tactile, audible and/or force feedback) to the operator such as to confirm a proper connection (e.g. confirm a fluid seal and/or sufficient electrical contact). In alternate embodiments, two or more of the attachable components of system 10 described herein can be provided in a pre-attached configuration, such as two or more components which are attached during a manufacturing process. System 10 can include one or more tools for attaching one component to another, not shown but such as tool 500 described in reference to FIG. 7 herebelow.

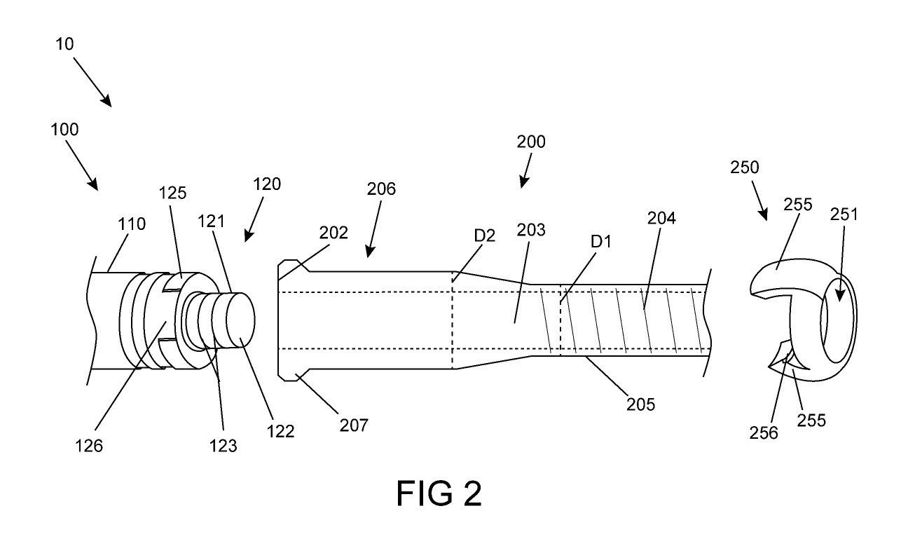

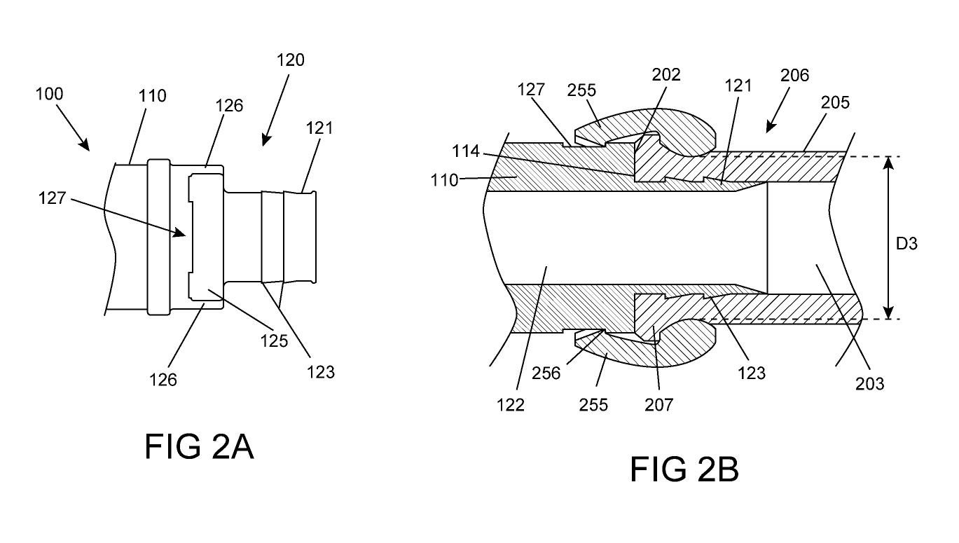

Inflow cannula assembly 200 and outflow graft assembly 300 each include a tubular portion constructed and arranged to transport fluid to and from, respectively, pump 100. Inflow cannula assembly 200 includes a tube, inflow conduit 205, comprising two ends, proximal end 201 and distal end 202, with lumen 203 therebetween. In operation, fluid flows from proximal end 201 to distal end 202. Inflow conduit 205 can comprise one or more flexible, biocompatible materials, such as one or more materials selected from the group consisting of: silicone; polyurethane, polyester, expanded polytetrafluoroethylene (ePTFE); and combinations of these. Inflow conduit 205 can comprise a single layer, multiple layer construction or a composite with a laminated construction. In some embodiments, lumen 203 of inflow conduit 205 comprises a diameter between approximately 2 mm and 10 mm. Inflow cannula assembly 200 comprises a connecting portion, inflow connector 220, near distal end 202, constructed and arranged to allow an operator to attach inflow cannula assembly 200 to housing 110, such as to create a sealed, fluid connection between inflow cannula assembly 200 and housing 110. In some embodiments, housing 110, inflow cannula assembly 200 and/or inflow connector 220 are constructed and arranged as is described in FIGS. 2 and 2A herebelow. In some embodiments, proximal end 201 is configured to be fluidly attached to a source of blood, such as a source of oxygenated blood, such as the left atrium or left ventricle of a patient, heart chamber HC as shown. In some embodiments, inflow cannula assembly 200 is configured as described in co-pending U.S. patent application Ser. No. 12/392,623, entitled “Devices, Methods and Systems for Establishing Supplemental Blood Flow in the Circulatory System”, filed Feb. 25, 2009, the disclosure of which is incorporated herein by reference in its entirety.

Outflow graft assembly 300 includes a tube, outflow conduit 305, comprising two ends, proximal end 301 and distal end 302, with lumen 303 therebetween. In operation, fluid flows from proximal end 301 to distal end 302. Outflow conduit 305 can comprise one or more flexible, biocompatible materials, such as one or more materials selected from the group consisting of: ePTFE; a polyester weave such as a sealed woven polyester material; and combinations of these. Outflow conduit 305 can comprise a single layer or multiple layer construction. In some embodiments, outflow conduit 305 comprises a three layer laminate comprising an inner layer, an outer layer, and a middle layer configured as a hydrostatic barrier. In some embodiments, lumen 303 comprises a diameter between 4 mm and 12 mm.

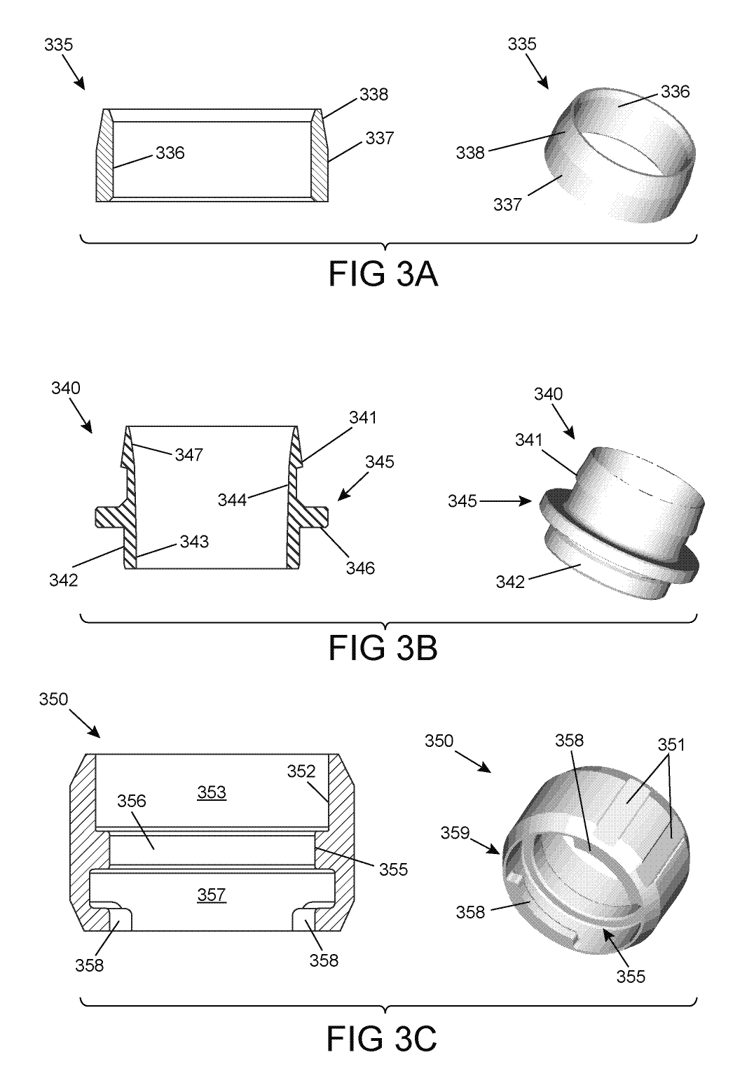

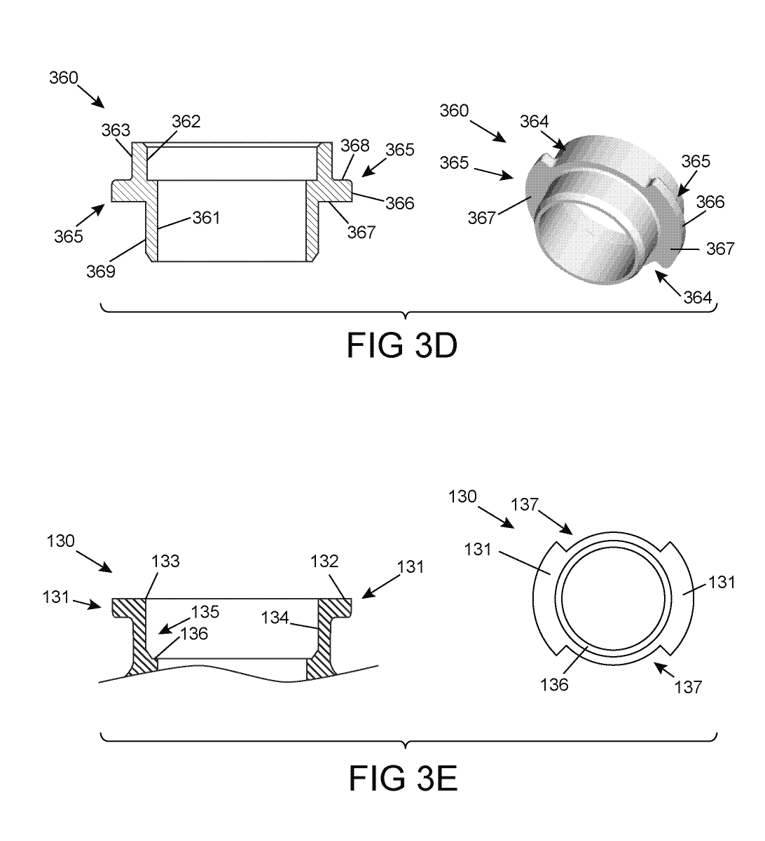

Outflow graft assembly 300 comprises a connecting portion, outflow connector 330, near proximal end 301, constructed and arranged to allow an operator to attach outflow graft assembly 300 to housing 110, such as to create a sealed, fluid connection between outflow graft assembly 300 and housing 110. In some embodiments, housing 110, outflow graft assembly 300 and/or outflow connector 330 are constructed and arranged as described in reference to FIGS. 3A-3E herebelow. In embodiments when inflow cannula assembly 200 is attached to a source of oxygenated blood, distal end 302 of outflow graft assembly 300 can be configured to be fluidly attached to a blood vessel, such as artery A as shown, such as via an anastomosis. In some embodiments, artery A comprises the subclavian artery. In some embodiments, outflow graft assembly 300 can comprise an anastomotic connector on its distal end 302, such as is described in the above incorporated U.S. Pat. No. 8,333,727.

System 10 further includes a control module 400, constructed and arranged to be operably attached to motor 115, such as via one or more conduits, such as to control the rotational velocity of fluid drive element 117. Control module 400 includes a power supply, such as a rechargeable battery. Control module 400 can include various electronic components, firmware, hardware and software, such as to allow variable rotational speed control of motor 115. In some embodiments, control module 400 is attached to pump 100 via one or more conduits comprising one or more electrical wires, such as wire bundles 101 and 401. Pump 100, inflow cannula assembly 200 and outflow graft assembly 300 are typically implanted in the patient while control module 400 can remain outside the patient such that wire bundles 101 and/or 401 travel from control module 400, passing through the skin of the patient, through tissue under the skin to the implanted pump 100. Alternatively or additionally, wire bundles 101 and/or 401 can comprise a functional conduit such as a rotational drive cable, such as a rotational drive cable operably engaged with and configured to rotate fluid drive element 117. Alternatively or additionally wire bundles 101 and/or 401 can comprise a fluid delivery tube constructed and arranged to deliver hydraulic and/or pneumatic fluid to an assembly of pump 100, such as to cause operation (e.g. rotation) of motor 115 without transferring electrical energy.

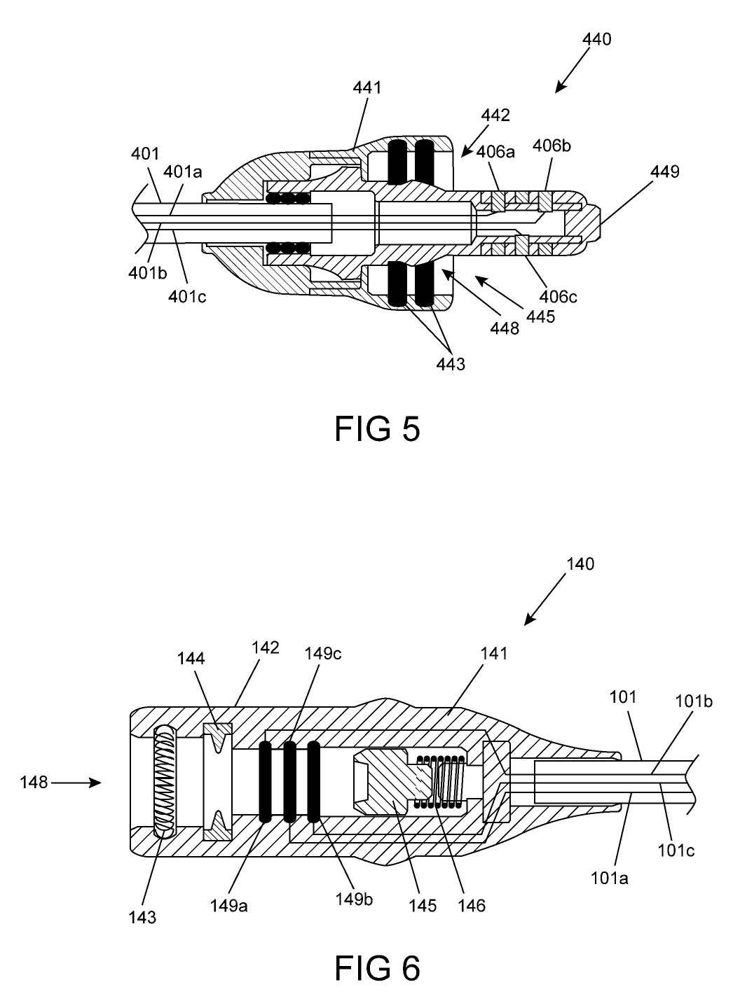

Wire bundle 401 can be configured to allow an operator to attach wire bundle 401 to control module 400, such as by inserting connector 402 of wire bundle 401 into an electrical connection port, jack 411. Jack 411 is configured to electrically connect wire bundle 401 to one or more electronic components internal to control module 400. Wire bundle 401 can further include an implantable connector 440, positioned on the end opposite from connector 402. Pump 100 can include wire bundle 101, with an implantable connector 140 on its end. Connectors 140 and 440 can be constructed and arranged to allow an operator to make a functional connection between control module 400 and pump 100, such as to create a sealed, electrical connection between wire bundle 401 and wire bundle 101. In some embodiments, housing 110 can comprise a portion of implantable connector 140, such that wire bundle 101 is contained within housing 110 or eliminated. In the illustrated embodiment, wire bundle 101 is fixedly attached to housing 110 and to implantable connector 140, such that implantable connector 140 can be manipulated (e.g. connected or disconnected from implantable connector 440) by an operator without the need to reposition or otherwise move housing 110. In some embodiments, implantable connectors 140 and 440 are configured as those described in reference to FIGS. 5 and 6, respectively, herebelow.

System 10 can include a tissue tunneling component, tunneler 403, configured to removably attach to connector 402 and facilitate a surgical technique used to advance connector 402 and the attached portion of wire bundle 401 through tissue (e.g. creating a tunnel for wire bundle 401 under the skin) of the patient. In some embodiments, tunneler 403 comprising a malleable elongate tube with a connector on one end and a sharpened tip on the other end. Tunneler 403 can be used to pull wire bundle 401 from a pump 100 implant location (e.g. the chest of the patient) to a wire bundle 401 skin exit location (e.g. a location proximate to a location where control module 400 is maintained during use, such as attached to a belt or a harness near the waist of the patient). In an alternative embodiment, implantable connector 440 can be configured to attach to tunneler 403, such that wire bundle 401 can be tunneled from a skin exit location to a pump 100 implant location (i.e. the opposite direction from that described hereabove). In some embodiments, tunneler 403 can create a fluid tight, or otherwise contamination preventing seal around its attached connector, such as to prevent body fluids or other material from contacting interface components of the connector, which otherwise can compromise an electrical or other connection.

System 10 can include one or more clamps, such as one or more tubing and/or vessel clamps (e.g. a Kelly clamp) used to temporarily compress an artificial tube and/or a blood vessel to reduce or prevent flow within the tube and/or vessel. System 10 can include clamp 209 as shown, such as a clamp used to restrict flow in inflow conduit 205 of inflow cannula assembly 200. Clamp 209 can be used to prevent undesired fluid flow through inflow conduit 205 from (and/or to) the patient's heart chamber HC, such as before inflow cannula assembly 200 is fluidly attached to pump 100 and/or before pump 100 is fluidly connected to artery A. System 10 can include clamp 309a as shown, such as a clamp used to restrict flow in outflow conduit 305 of outflow graft assembly 300. Clamp 309a can be used to prevent undesired fluid flow through outflow conduit 305, such as undesired blood flow from artery A. Clamp 309a can be used to prevent undesired blood flow during the anastomosis of outflow graft assembly 300 to artery A and/or before attachment of outflow graft assembly 300 to pump 100. Clamp 309b can be used to prevent undesired blood flow through artery A, such as during the creation of an anastomosis to outflow conduit 305. Clamp 309b can be temporarily loosened such as to confirm the integrity of the anastomosis of outflow graft assembly 300 to artery A (i.e. to confirm no leaks are present).

During implantation of pump 100, inflow cannula assembly 200 and outflow graft assembly 300, the blood flow pathways are primed with fluid such as blood and/or saline. In some embodiments, prior to attaching inflow cannula assembly 200 to pump 100, outflow graft assembly 300 is anastomosed to artery A and outflow graft assembly 300 is attached to pump 100. Blood from artery A is allowed to flow (in a retrograde direction) through outflow conduit 305 and pump 100, priming those flow pathways prior to the attachment of inflow cannula assembly 200 to pump 100. Inflow cannula assembly 200 can be anastomosed to heart chamber HC prior to attachment of outflow graft assembly 300 to artery A. In some embodiments, one or more fluid pathways of system 10 can include a de-gassing port or plug, not shown but configured to allow an operator to remove air or other gas from a fluid pathway of system 10. In some embodiments, saline is delivered to the distal end 202 of inflow cannula assembly 200, displacing any air or other gas in lumen 203 in order to prime inflow cannula assembly 200 prior to attachment to pump 100.

System 10 can be constructed and arranged such that pump 100, inflow cannula assembly 200, outflow graft assembly 300, wire bundle 401, artery A and heart chamber HC can be attached to a corresponding element in any order. Fluid connections between system 10 components and flow conduits of the patient are constructed and arranged to minimize at least one of: turbulence; flow stagnation; and a low-flow location. One or more clamps, such as clamp 209, 309a an 309b described herebelow, can be used to compress one or more flexible portions of a fluid pathway component of system 10, to reduce or prevent flow through that component. Clamps can also be used on a blood vessel, such as artery A.

In one embodiment, pump 100, inflow cannula assembly 200, outflow graft assembly 300 and wire bundle 401 are implanted and operably attached in one or more of the following steps. A subcutaneous pocket can be created to surround pump 100. Inflow cannula assembly 200 is anastomosed to heart chamber HC such as through a mini-thoracotomy. Clamp 209 can be applied to inflow conduit 205 to prevent undesired flow within inflow cannula assembly 200. Inflow cannula assembly 200 can be tunneled through the patient's ribs (under the skin) to the subcutaneous pocket. Clamp 309b can be attached to artery A, after which outflow graft assembly 300 is anastomosed to artery A. Clamp 309a can be positioned on outflow conduit 305 during the anastomosis. After the anastomosis, clamp 309b can be loosened to confirm the absence of leaks at the anastomosis, and subsequently re-clamped. After the anastomosis, outflow graft assembly 300 is attached to inflow port 120 of pump 100 and one or more of clamps 309a and 309b are released such that blood flows in a retrograde direction through outflow graft assembly 300 and pump 100, priming the fluid pathways of each, without any need to rotate fluid drive element 117 (e.g. without operating motor 115). After priming, one or more of clamps 309a and 309b can be re-clamped (e.g. clamp 309a) to maintain primed state. Saline can be introduced into the distal end 202 of inflow cannula assembly 200, such as when clamp 209 is clamping inflow conduit 205, displacing any air or other gas from the distal portion of inflow conduit 205. Subsequently inflow cannula assembly 200 can be attached to inflow port 120 of pump 100, and one or more of clamps 209, 309a and/or 309b are released. Connector 440 is attached to connector 140 of pump 100. Prior to or after attachment of connector 440 to connector 140, wire bundle 401 can be attached to tunneler 403 and tunneled under the patient's skin as described hereabove. Wire bundle 401 is subsequently attached to control module 400, at a location external to the patient (i.e. wire 401 bundle passes through the patient's skin).

Referring now to FIG. 2, a perspective view of an inflow cannula assembly positioned for attachment to an inflow port of a fluid pump is illustrated, consistent with the present inventive concepts. System 10 can include components similar to the components of system 10 of FIG. 1, such as pump 100, inflow port 120 and inflow cannula assembly 200 of FIG. 1. In the embodiment of FIG. 2, a particular configuration of inflow port 120 and inflow cannula assembly 200 is illustrated, allowing an operator to simply and rapidly create a fluid connection between pump 100 and inflow cannula assembly 200, such as a connection made during a sterile clinical procedure by a surgeon.

Inflow port 120 includes an elongate tubular segment, neck 121, protruding from housing 110 of pump 100. Neck 121 surrounds an inflow lumen, lumen 122, which is fluidly connected to an internal chamber of pump 100, such as chamber 111 of FIG. 1. Neck 121 further includes one or more cannula engaging radial outward facing projections, barbs 123. Inflow cannula assembly 200 comprises inflow conduit 205, with lumen 203 extending from its proximal end (not shown but such as proximal end 201 of FIG. 1) to its distal end 202. At least a portion of inflow cannula assembly 200 can include a radial reinforcing element, such as spring 204 configured to prevent kinking, twisting and/or other undesired flexing of inflow cannula assembly 200. Inflow cannula assembly 200 further comprises a portion having a thickened wall, distal segment 206, positioned near its distal end 202, with outer diameter D2 configured to provide enhanced compressibility. In some embodiments, the remaining portion of inflow cannula assembly 200 comprises a reduced diameter, diameter D1. In some embodiments, spring 204 can terminate prior to distal segment 206 (as shown), such as to avoid interfering with the compression connection between distal segment 206 and neck 121.

The distal portion of lumen 203 (i.e. the portion surrounded by distal segment 206) is configured to slidingly receive neck 121, including barbs 123, of inflow port 120, such that inflow conduit 205 becomes fluidly connected to lumen 122 of pump 100. Barbs 123 are configured to enhance the fluid seal and/or resist undesired slippage of inflow conduit 205 from neck 121. Distal segment 206 can be configured to stretch or otherwise adapt during its sliding placement over neck 121. Inflow cannula assembly 200 further includes an inflow connector, such as inflow connector 220 of FIG. 1, comprising collar 250. Collar 250 is constructed and arranged to further secure inflow conduit 205 to pump 100 such as via a compression fitting. Collar 250 includes an opening 251 as well as two or more arms 255. Arms 255 each include radially inward facing projections, flanges 256. Opening 251 is sized to allow collar 250 to slidingly receive inflow cannula assembly 200, such as to be placed over inflow conduit 205 before or during a clinical procedure, such as a sterile surgical implantation procedure performed by an operator such as a surgeon. In some embodiments, the length of inflow conduit 205 can be reduced (e.g. shortened) by a clinician during implantation of inflow cannula assembly 200, such as by cutting off a segment of inflow conduit 205 from its proximal and/or distal end.

Referring additionally to FIG. 2A, a side view of inflow port 120 of FIG. 2 is illustrated, consistent with the present inventive concepts. Housing 110 includes multiple radially outward facing projections, projections 125, proximate inflow port 120. Positioned between projections 125 are recesses 126, positioned and configured to allow arms 255 of collar 250 to pass through recesses 126 and extend beyond projections 125. Each projection 125 can include a recessed capture area, notch 127. Each notch 127 can be configured to receive and frictionally engage (e.g. capture) a flange 256 of collar 250 (e.g. after a rotation of collar 250) as described in detail herebelow.

Referring additionally to FIG. 2B, a side sectional view of inflow cannula assembly 200 of FIG. 2 attached to inflow port 120 of FIGS. 2 and 2A is shown, consistent with the present inventive concepts. Inflow cannula assembly 200 has been attached to inflow port 120 by advancing inflow conduit 205 over neck 121, aligning collar 250 (already placed over inflow conduit 205) with recesses 126, advancing collar 250 toward housing 110, and subsequently rotating collar 250 to engage notches 127. Collar 250 is configured to, while sliding distally across distal segment 206 of inflow cannula assembly 200, engage housing 110 by frictionally engaging projections 125. Arms 255 are first slidingly received by housing 110, passing distal to projections 125. Flanges 256 are positioned beyond projections 125 after collar 250 has been slid a sufficient distance towards distal end 202 such that collar 250 compresses inflow cannula assembly 200 around neck 121 of inflow port 120. Collar 250 is configured to then be rotated; such that flanges 256 engage projections 125, affixing collar 250 to housing 110. In some embodiments, inflow cannula assembly 200 is constructed and arranged and configured to be attached to inflow port 120 as described immediately herebelow.

Inflow cannula assembly 200 comprises an inner diameter D1 as shown. Distal segment 206 comprises an outer diameter D2 as shown. Opening 251 of collar 250 comprises an inner diameter D3 as shown. Diameter D3 of opening 251 can be configured to be greater than or equal to diameter D1 of inflow cannula assembly 200, such that collar 250 can be advanced freely along the length of inflow cannula assembly 200. Diameter D3 can be further configured to be less than diameter D2 of distal segment 206 of inflow cannula assembly 200, such that collar 250 compresses at least a portion of distal segment 206 when a portion of distal segment 206 is positioned within collar 250 (e.g. as shown in FIG. 2B). Distal segment 206 can also include a flanged end, lip 207, at distal end 202 of inflow cannula assembly 200. Lip 207 can be configured to have an outer diameter greater than the diameter of opening 251 of collar 250, diameter D3, such as to prevent lip 207 from undesirably passing through opening 251 collar 250 (e.g. such that collar 250 captures lip 207 and maintains security of attachment of inflow cannula assembly 200 to pump 100).

After neck 121 has been slidingly received by lumen 203 of inflow cannula assembly 200, (e.g. such that neck 121 is surrounded by inflow conduit 205 and lip 207 is positioned in proximity with surface 114 of housing 110 as shown in FIG. 2B) collar 250 is configured to slide distally (i.e. to the left of the page) along inflow cannula assembly 200, toward pump 100. Collar 250 compresses distal segment 206, such that the portion of lumen 203 within the compressed portion of distal segment 206 is compressed, or otherwise sealed around neck 121 of pump 100. This compression segment, along with barbs 123, helps ensure a proper, leak-free, fluid connection between lumens 203 and 122 of inflow cannula assembly 200 and pump 100, respectively.

Pump 100 can include features to lock, or otherwise secure collar 250 to pump 100, such as to maintain the position of collar 250 after a fluid seal has been achieved, such as the locking provided by the capture of flanges 256 by notch 127 as described above. In some embodiments, collar 250 is constructed and arranged to be rotated by a wrench or other tool, such as the torque-limiting wrench, tool 500 of FIG. 7 described herebelow.

As described hereabove, projections 125 can include one or more notches 127, configured to engage flanges 256 of collar 250 to maintain a secure connection of inflow cannula assembly 200 to pump 100. In some embodiments, lip 207 of inflow cannula assembly 200 can be configured to oppose collar 250, such as to provide a force configured to oppose the movement of collar 250 distally along the distal segment 206 of inflow cannula assembly 200. In these embodiments, notches 127 slidingly receive flanges 256, for example when collar 250 has been rotated 90 degrees to engage projections 125, and the opposing force provided by compression of lip 207 pushes collar 250 proximately, such that flanges 256 are biased to remain seated in notches 127. This engagement of flanges 256 within notches 127 can be configured to provide an audible or tactile feedback, such as a clicking noise made during the full engagement of collar 250, such as to provide a clinician with feedback that a proper connection has been made.

In some embodiments, inflow cannula assembly 200 is attached to pump 100 by hand (e.g. finger-tightening), and in other embodiments a tool is used, such as a torque-limiting wrench such as tool 500 of FIG. 7 described herebelow. In some embodiments, an anti-rotation, axial slip prevention and/or other retention force is provided by the compression of a portion of distal segment 206 against collar 250. In some embodiments, an anti-rotation force is provided by the engagement of collar 250 with an anti-rotating element of housing 110, such as notches 127. Proper engagement of collar 250 with housing 110 can be identified via audible feedback (e.g. a clicking sound provided when collar 250 engages notches 127), or visible feedback (e.g. when collar 250 is in a proper rotational position). In some embodiments, collar 250 and neck 121 are designed to minimize trauma to inflow conduit 205, such as trauma causing during or after attachment of inflow cannula assembly 200 to pump 100. In some embodiments, trauma to inflow conduit 205 is reduced or eliminated by distributing the stress across the contacting surfaces of collar 250, inflow conduit 205 and/or neck 121.

Referring now to FIGS. 3A-3D, side sectional and perspective views of the components of an outflow graft assembly including an independently rotatable collar are illustrated, consistent with the present inventive concepts. Referring also to FIG. 3E, a side sectional view of an inflow port of a fluid pump is illustrated, for attachment to the outflow graft assembly connection components of FIGS. 3A-3D. The side sectional views of the components of FIGS. 3A-3E are oriented such that the distal ends and/or surfaces of the components are oriented towards the top of the page and the proximal ends and/or surfaces are oriented towards the bottom of the page. In operation, fluid flows from each proximal end to each distal end. The components of FIGS. 3A-3E can comprise one or more materials similar to the materials of housing 110 of FIG. 1, such as materials selected from the group consisting of: metals such as stainless steel or titanium; plastics such as one or more rigid biocompatible plastics; and combinations of these.

FIG. 3A illustrates a compression ring, ring 335. Ring 335 comprises inner and outer surfaces, surfaces 336 and 337 respectively. Outer surface 337 includes a chamfer, chamfer 338, positioned at the distal end of ring 335. Inner surface 336 can comprise chamfered, rounded or otherwise contoured edges at the proximal and/or distal ends of ring 335, such as to avoid damaging an outflow graft conduit, such as outflow conduit 305 of FIG. 1.

FIG. 3B illustrates a compression fitting, fitting 340. Fitting 340 comprises a tubular body with a full circumferential radially outward facing projection, flange 345, positioned between the proximal and distal ends of fitting 340. Flange 345 includes a proximal surface, surface 346. Flange 345 can comprise chamfered, rounded or otherwise contoured edges. The distal portion of fitting 340 includes a cannula engaging projection, barb 341, configured to frictionally engage a tubular conduit, such as outflow conduit 305 of FIG. 1. In some embodiments, fitting 340 includes two or more cannula engaging projections extending radially from the tubular structure of fitting 340, such as two or more barbs 341. The proximal portion of fitting 340 includes a threaded segment, threads 342. Threads 342 comprise male threads (i.e. outward facing threads). Fitting 340 further comprises an inner surface, surface 343, comprising a first taper, taper 344, extending from the proximal end of fitting 340, and a second taper, taper 347, extending from taper 344 to the distal end of fitting 340. In some embodiments, first taper 344 comprises a first taper angle from a central axis of fitting 340 and second taper 347 comprises a second, larger taper angle from the central axis of fitting 340. In some embodiments, first taper 344 comprises a linear taper. In some embodiments, taper 347 comprises a curvilinear taper. Tapers 344 and 347 can be sized to provide a continuously increasing inner diameter of fitting 340 and/or they can be configured to minimize or prevent turbulent flow within the associated outflow graft assembly.

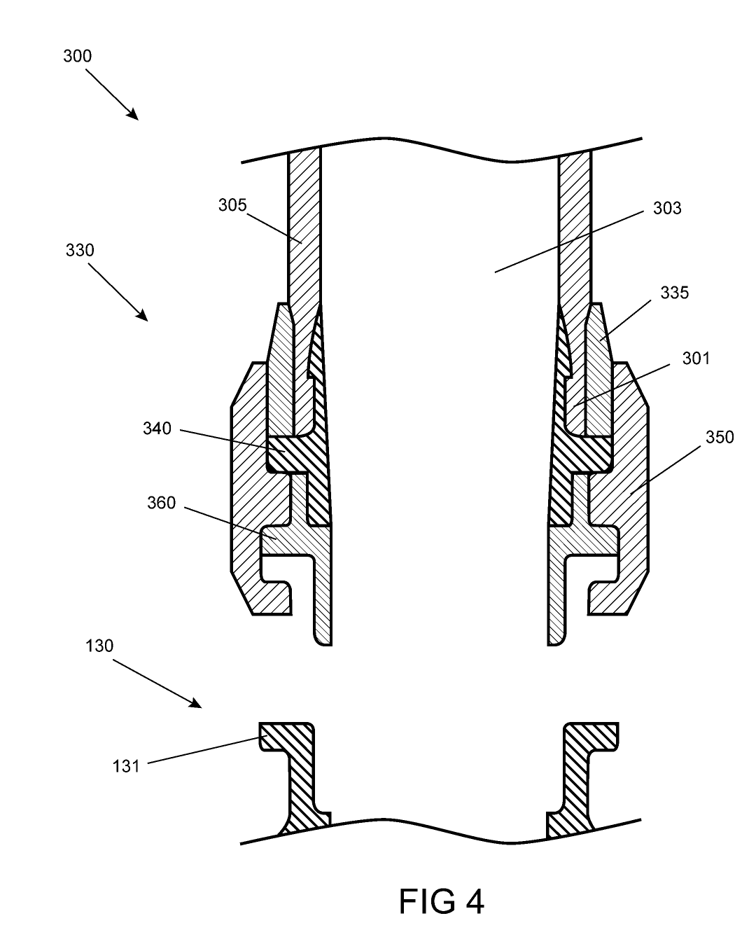

FIG. 3C illustrates an outflow connector for an outflow graft assembly comprising collar 350. Collar 350 comprises a tubular body sized to surround a flow conduit. Collar 350 can be constructed and arranged to be independently rotatable. The outer and inner surface edges of collar 350 can comprise chamfered, rounded and/or otherwise contoured edges, such as to avoid damage to one or more mating components. The outer surface of collar 350 includes multiple axially aligned recesses, grooves 351 as shown, configured to assist an operator in the rotation of collar 350, such as by a finger turning operation, or with the use of a tool such as a wrench, such as the torque-limiting wrench, tool 500 described in reference to FIG. 7 herebelow. Collar 350 includes a full circumferential radially inward facing projection, flange 355, positioned between the distal and proximal ends of collar 350. Flange 355 comprises an inner surface 356, and can further include chamfered, rounded and/or otherwise contoured edges. Flange 355 is positioned at the proximal end of distal capture chamber 353, which is surrounded by inner walls 352 of collar 350. Distal capture chamber 353 is constructed and arranged to slidingly receive at least a portion of fitting 340, such as is described in reference to FIG. 4 herebelow.

Flange 355 is further positioned at the distal end of proximal capture chamber 357. Collar 350 includes multiple partial circumferential radially inward facing projections, projections 358, positioned near the proximal end of collar 350 as shown. Gaps 359 each comprise a recess positioned between projections 358, also as shown. Projections 358 are positioned at the proximal end of proximal capture chamber 357. Projection 358 can comprise chamfered, rounded and/or otherwise contoured edges and/or corners.

FIG. 3D illustrates an adapter, adapter 360, also comprising a tubular structure. The distal portion of adapter 360 includes a threaded segment, threads 362. Threads 362 are configured as female threads, such as to rotatably engage male threads 342 of fitting 340, such as is described in reference to FIG. 4 herebelow. Proximal to threads 362, adapter 360 comprises an inner surface 361. Inner surface 361 can comprise a smaller diameter than threads 362, as shown. Adapter 360 further includes one or more partial circumferential radially outward facing projections, projections 365, positioned between the proximal and distal ends of adapter 360. Projections 365 include outer, proximal, and distal surfaces, surfaces 366, 367 and 368, respectively as shown. Positioned between each projection 365 is a recess, opening 364 as shown. Projections 365 and openings 364 are configured such that in the proper orientation, adapter 360 can be slidingly received into proximal capture chamber 357 of collar 350, such as is described in reference to FIG. 4 herebelow.

Adapter 360 comprises outer surface 363 at its distal end, surrounding threads 362. Outer surface 363 is constructed and arranged to be slidingly received by inner surface 356 of collar 350, and is also configured to allow collar 350 to rotate freely and smoothly about outer surface 363 (i.e. the components are sized such that the components outer and inner diameters are similar but provide a sufficient gap or are otherwise configured to allow smooth rotation without binding). Adapter 360 further comprises outer surface 369 at its proximal end, surrounding inner surface 361. Surfaces 369, 361 and contours therebetween at the proximal end of adapter 360 are configured to mate with an outflow port of a fluid pump, such as outflow port 130 of FIGS. 1 and 3E, such as to create a fluid tight seal and to optimize flow conditions (e.g. the components are configured to fit together snugly and result in a flush, relatively continuous inner surface configured to minimize turbulence, flow stagnation or low-flow locations).

FIG. 3E illustrates side sectional and end views of the outflow port 130 of a fluid pump, such as pump 100 of FIG. 1. Outflow port 130 includes one or more partial circumferential radially outward facing projections, projections 131, positioned at its distal end. Projections 131 define multiple openings 137, such that projections 131 and openings 137 can be aligned with gaps 359 and projections 358, respectively, such that outflow port 130 can be to be slidingly received by proximal capture chamber 357 of collar 350. In some embodiments, projections 131 are sized and positioned similar to projections 365 of adapter 360 of FIG. 3D. Projections 131 comprise distal surfaces 132, which are flush with the full circumferential distal surface, surface 133, of outflow port 130. Distal surfaces 132 and 133 are configured to oppose surface 367 of adapter 360, such as when the assembled outflow graft assembly (e.g. outflow graft assembly 300 of FIG. 4) is operably attached to outflow port 130. Outflow port 130 further includes recess 135, ledge 136 and inner surface 134. Recess 135 is configured to slidingly receive adapter 360, such that the proximal surface of adapter 360 opposes ledge 136, such that a fluid seal is created between outflow port 130 and the assembled outflow graft assembly.

Referring additionally to FIG. 4, a side sectional view of an end of an outflow graft assembly comprising the components of FIGS. 3A-3D is illustrated, including an independently rotatable collar and positioned for attachment to outflow port 130 of FIG. 3E, consistent with the present inventive concepts. Collar 350 is constructed and arranged to allow an operator to connect outflow conduit 305 to outflow port 130. Components of outflow connector 330 can be provided in the pre-assembled configuration shown in FIG. 4, such as when the components are assembled during a manufacturing or post manufacturing process, such as a process as described herebelow. In some embodiments, collar 350 is pre-attached to outflow conduit 305 in a manufacturing process. Additionally or alternatively, one or more components can be assembled by a clinician, such as a surgeon during a sterile clinical procedure, for example to replace the connector on the end of a previously implanted cannula. Various tools can be used to assemble outflow connector 330, such as tools selected from the group consisting of: a press, such as a hand-held hydraulic press constructed and arranged to secure ring 335, including outflow graft assembly 300, onto fitting 340; a spreader, such as a spreader configured to ease in the insertion of fitting 340 into outflow graft assembly 300; an alignment device, such as an alignment device constructed and arranged to assist in the assembly of the various components; and combinations of these.

Outflow connector 330 can be attached to outflow graft assembly 300 as shown in FIG. 4, such as an attachment made during a manufacturing process. Various tools can be used to aid in the assembly process as described herein. Proximal end 301 of outflow graft assembly 300 is configured to be slidingly received by ring 335, such that ring 335 is slid towards the medial portion of outflow graft assembly 300. Proximal end 301 is configured to then slidingly receive fitting 340, such that barb 341 frictionally engages outflow graft assembly 300. A spreader tool can be used to dilate proximal end 301 to ease in the insertion of fitting 340. Ring 335 is configured to then be slid distally over outflow graft assembly 300, compressing proximal end 301 between inner surface 336 and fitting 340. Taper 347 is configured to create a smooth transition between fitting 340 and outflow graft assembly 300, such as to minimize the creation of turbulence, or flow stagnation within lumen 303.

Distal capture chamber 353 is configured to slidingly receive the proximal end of the assembly comprising outflow graft assembly 300, fitting 340 and ring 335, such that flange 345 and outer surface 337 oppose inner wall 352, and proximal surface 346 opposes the distal surface of flange 355. As described hereabove, adapter 360 is configured to be inserted into proximal capture chamber 357, and be further slidingly received by inner surface 356 of collar 350. Threads 342 and 362 are rotatably engaged, such that fitting 340 and adapter 360 are fixedly attached, creating a fluid seal between the two components. Inner surfaces 343 and 361 are configured to create a flush interface, such as to minimize or prevent turbulence or low-flow locations within system 10. In the assembled configuration shown in FIG. 4, the surfaces opposing any portion of collar 350 are configured to allow collar 350 to independently rotate freely and smoothly about the distal portion of outflow graft assembly 300, such as to allow a simple, low-torque rotation of collar 350, such as to simplify alignment of collar 350 for attachment to outflow port 130 as described immediately herebelow.

Outflow connector 330 is constructed and arranged to be rotatably connected to outflow port 130 during a clinical procedure. Collar 350 can be rotated about outflow graft assembly 300, such that projections 131 align with gaps 359, and adapter 360 can be slidingly received by outflow port 130. Proximal surface 367 is configured to oppose surfaces 132 and 133, such that projections 131 are within proximal capture chamber 357. Collar 350 is configured to be subsequently rotated, such as to be rotated 90°, such that projections 131 are locked within proximal capture chamber 357. This rotation secures outflow connector 330 to outflow port 130, creating a fluid seal and minimizing turbulence proximate outflow port 130 and/or within lumen 303. In some embodiments, the distal end of outflow conduit 305 (e.g. distal end 302 of FIG. 1) is anastomosed to a body conduit, such as an artery, prior to attaching outflow graft assembly 300 to outflow port 130. In some embodiments, outflow graft assembly 300 is constructed and arranged to allow relatively free (e.g. low-torque) rotation of outflow conduit 305 after collar 350 is attached to port 130 of pump 100. In some embodiments, outflow graft assembly 300 is constructed and arranged to allow relatively free (e.g. low-torque) rotation of collar 350 prior to, during and/or after the creation of the anastomosis. In these embodiments, one or more components of outflow graft assembly 300 and/or pump 100 can be constructed and arranged to be further tightened to prevent this low-torque rotation. Adequacy of this further tightening can be accomplished through the use of a torque-limiting wrench, such as tool 500 of FIG. 7 and/or by feedback, such as audible (e.g. a clicking sound) or tactile feedback (e.g. the slipping of a torque-limiting wrench) as described herein. In some embodiments, the length of outflow conduit 305 can be reduced (e.g. shortened) by a clinician during implantation of outflow graft assembly 300, such as by cutting off a segment of outflow conduit 305 from its proximal and/or distal end.

Referring now to FIG. 5, a side sectional view of an implantable male electrical connector is illustrated, consistent with the present inventive concepts. Wire bundle 401 (e.g. wire bundle 401 of control module 400 of FIG. 1) can terminate in an implantable male connector, connector 440 as shown. In this embodiment, wire bundle 401 comprises three conductors, 401a-401c. Connector 440 is constructed and arranged to provide an implantable, contamination-resisting connection for wires 401a-401c to operably (e.g. electrically) connect to three corresponding wires, such as three wires within a second wire bundle, such as when connector 440 connects to a female connector 140 of FIG. 1 and/or FIG. 6. In alternate embodiments, connector 440 can be configured to be a female connector, while connector 140 of pump 100 is configured to be a male connector. In other alternate embodiments, connector 440 can be attached to wire bundle 101 of FIG. 1, while connector 140 of FIG. 1 and/or FIG. 6 is attached to wire bundle 401.

Connector 440 comprises housing 441, surrounding at least a portion of connector 440, including opening 442, which also surrounds at least a portion of connector 440. Housing 441 includes frictionally engaging surfaces, O-rings 443, configured to engage a portion of a surface of a mating connector as described herebelow in reference to FIG. 6, such as to create a fluid tight or otherwise contamination resistant implantable connection. O-rings 443 can be constructed and arranged to provide a wiping (e.g. a cleaning) of one or more components passing therethrough. Connector 440 also comprises an electrical connector portion, jack 445, including distal tip 449, shown protruding from housing 441. In some embodiments, jack 445 can be entirely contained within opening 442. Jack 445 includes one or more electrical contacts, such as rings 406a-406c, which are electrically connected to wires 401a-401c respectively. Wires 401a-401c can be connected to rings 406a-406c via a solder joint, weld, crimp, or the like. Jack 445 further includes flared portion 448, configured to engage a portion of a surface of a mating connector, such as the connector described in reference to FIG. 6 herebelow.

Referring additionally to FIG. 6, a side sectional view of an implantable female electrical connector configured to electrically connect with the male implantable electrical connector 440 of FIG. 5 is illustrated, consistent with the present inventive concepts. Wire bundle 101 can terminate in an implantable connector, connector 140 as shown. Wire bundle 101 can be constructed and arranged in a similar manner to wire bundle 101 of pump 100 of FIG. 1. Wire bundle 101 comprises three conductors 101a-101c. Connector 140 is constructed and arranged to operably connect to an opposing connector, such as connector 440, such that conductors 101a-101c are electrically connected to wires 401a-401c, respectively.

Connector 140 comprises housing 141, including an opening 148. Opening 148 is constructed and arranged to slidingly receive jack 445 of connector 440, such that rings 406a-406c are positioned within connector 140 and are aligned with corresponding electrical contacts, contacts 149a-149c. Contacts 149a-149c are electrically connected to wires 101a-101c, respectively. Wires 101a-101c can be connected via a solder joint, weld, crimp, or the like.

Connector 140 can further include components constructed and arranged to align and stabilize jack 445 within opening 148. Components can include tensioning spring 143, alignment ring 144, and a tip guide assembly, including capture port 145 and spring 146. Spring 143 is configured to allow connector 140 to engage flared portion 448 of jack 445, such as to provide a tactile and/or audible feedback to the operator (e.g. the implanting surgeon), confirming proper engagement, as well as to retain jack 445 within opening 148. Alignment ring 144 is configured to center jack 445 within opening 148, as well as make contact with flared portion 448 to prevent jack 445 from being inserted too far into opening 148. Capture port 145, including spring 146, is configured to capture distal tip 449 of jack 445, and provide positive force feedback to the operator while connecting connectors 440 and 140.

Housing 141 includes an outer surface 142, configured to slidingly engage O-rings 443, within opening 442 of connector 440. O-rings 443 create a fluid tight or otherwise secure, reliable, implantable connection interface while engaged with outer surface 142.

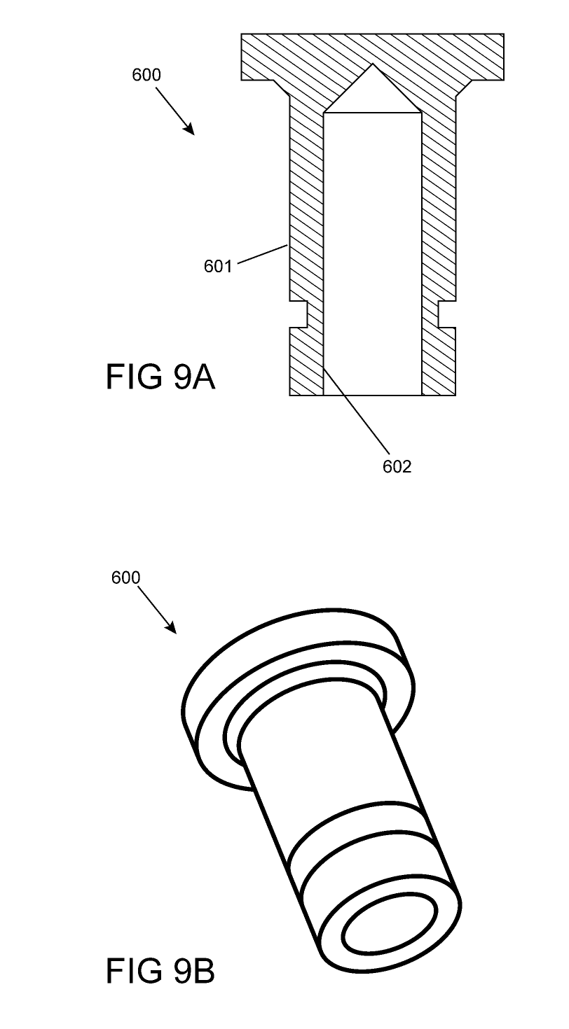

Connector 440 of FIG. 5 and/or connector 140 of FIG. 6 can be provided with fluid absorbing plugs constructed and arranged to dry one or more electrical or other surfaces, such as plug 600 described in reference to FIGS. 9A and 9B herebelow. Housing 441 of connector 440 and/or housing 141 of connector 140 can comprise a polymer or other material configured to prevent tissue in-growth, such as to ease removal of the connector during an explantation procedure (e.g. ease dissection and separation from tissue after the connector has been implanted in the body for an extended time period).

Referring now to FIGS. 7A and 7B, a side view of a tool for attaching two components of a fluid flow system, consistent with the present inventive concepts. Tool 500 comprises a wrench configured to grip a rotatable component (e.g. grip opposing surfaces of a nut or collar) and apply a limited torsional force to the rotatable component. Tool 500 comprises a handle 505 and a distal portion 509. Distal portion 509 includes arms 501 configured to frictionally engage the rotatable component, apply a torsional force to rotate and/or tighten an assembly in which the component is attached, and subsequently disengage when a threshold torsional force is reached. Arms 501 can include a recess or other object-gripping element, such as curvilinear recess 502 positioned on the component engaging surface of arms 501 as shown.

Tool 500 can be configured such that distal portion 509 disengages from handle 505 when sufficient torque (e.g. a maximum torque) is applied to a rotatable component by arms 501. In the embodiment shown in FIGS. 7A and 7B, tool 500 includes a disengaging assembly 510 including capturing hinge 503, capture port 504, channel 506, spring, 507 and locking element, a tensioning ball 508. Hinge 503 rotatably attaches distal portion 509 to handle 505. Channel 506 is positioned in handle 505 and surrounds spring 507. Ball 508 is positioned at an end of spring 507, and is securingly captured by capture port 504, locking handle 505 and distal portion 509 in the linear configuration shown in FIG. 7A. When a rotating force is applied to handle 505, the force is transmitted to arms 501 until a threshold torque level is achieved. The components of disengaging assembly 510 are constructed and arranged such that at this threshold torque level (e.g. sufficient torque to tighten and/or lock an assembly of system 10), ball 508 compresses spring 507 such that ball 508 disengages with capture port 504 (e.g. ball 508 translates away from capture port 504, deeper into channel 506) allowing distal portion 509 to rotate about a center of hinge 503, and preventing additional torque being applied by arms 501.

Referring now to FIG. 7B, ball 508 has compressed spring 507 and exited capture port 504 such that distal portion 509 of tool 500 has rotated and additional force applied to handle 505 will not be transmitted to arms 501 and any rotatable element engaged by arms 501.

In some embodiments, tool 500 can be constructed and arranged to perform a limited force rotation (e.g. a tightening) of a component of an inflow cannula assembly, such as inflow cannula assembly 200 of FIG. 1 or 2. In some embodiments, tool 500 can be constructed and arranged to perform a limited force rotation of a component of an outflow graft assembly, such as outflow graft assembly 300 of FIG. 1 or 4. In some embodiments, tool 500 can be constructed and arranged to perform a limited force rotation of an electrical connector, such as an electrical connector connecting pump 100 to control module 400, each of system 10 of FIG. 1

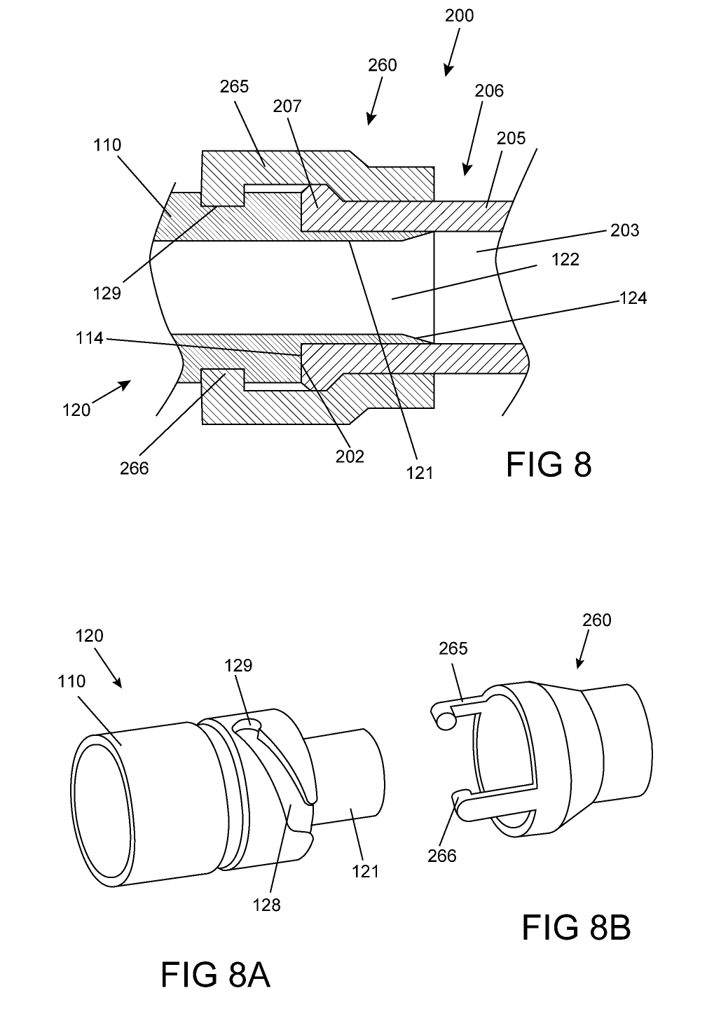

Referring now to FIG. 8, a side sectional view of a distal end of an inflow cannula assembly attached to an inflow port of a fluid pump is illustrated, consistent with the present inventive concepts. Inflow conduit 205 and a bayonet-style connector, collar 260, each of inflow cannula assembly 200, have been placed over neck 121 of inflow port 120. The geometry (e.g. inner diameter and wall thickness) and materials of construction of inflow conduit 205 are chosen such that inflow conduit 205 forms a fluid seal with neck 121 as inflow conduit 205 is slidingly engaged over neck 121. Inflow conduit 205 has been positioned such that distal end 202 of inflow conduit 205 is in contact with surface 114 of housing 110. In some embodiments, neck 121 comprises an inner surface with a taper, such as taper 124 configured to prevent turbulence and low-flow locations as fluid flows from lumen 203 of inflow conduit 205 into lumen 122 of neck 121. In the embodiments of FIGS. 8, 8A and 8B, collar 260, inflow conduit 205 and neck 121 can be dimensioned or otherwise configured to reduce or prevent compression of inflow conduit 205, as is described in detail herebelow. Distal segment 206 of inflow conduit 205 can include a radial flange on its distal end 202, lip 207 such as has been described in reference to FIGS. 2 and 2B hereabove.

Referring additionally to FIGS. 8A and 8B, perspective views of the inflow port 120 and the distal portion of inflow cannula assembly 200 of FIG. 8, respectively, are illustrated. Inflow port 120 and inflow cannula assembly 200 shown in FIGS. 8A and 8B, respectively, can be of a similar configuration to inflow port 120 and inflow cannula assembly 200 of FIG. 1. In the embodiment of FIGS. 8, 8A and 8B, housing 110 of pump 100 includes a recessed portion, channel 128, terminating in a retention portion, recess 129. Channel 128 is configured to slidingly receive a portion of collar 260. Collar 260 comprises one or more extending portions, arms 265, including radially inward facing projections, such as pins 266. Pins 266 are configured to be slidingly received by a recess, channel 128, such as when pins 266 are inserted into the opening of channel 128 and collar 260 is subsequently rotated (a clockwise rotation in the configuration shown). Full rotation of collar 260 causes pins 266 to engage recess 129. Arms 265, pins 266 and/or recesses 129 can be constructed and arranged to provide a retention force when pins 266 are positioned within recess 129, such as when recess 129 has a greater depth than channel 128 and arms 265 are biased to push pins 266 into the greater depth.

Collar 260 is further configured to frictionally engage at least a segment of inflow conduit 205. Lip 207, at the distal end 202 of inflow conduit 205 can comprise an outer diameter greater than the inner diameter of the proximal portion of collar 260, while the distal segment 206 of inflow conduit 205 comprises an outer diameter relatively similar to the inner diameter of the proximal portion of collar 260. Approximate matching of the outer diameter of distal segment 206 with the inner diameter of collar 260 prevents compressive or otherwise unwanted forces from being applied to inflow conduit 205 and/or neck 121. Collar 260 frictionally engages lip 207, affixing inflow conduit 205 to inflow port 120 of housing 110, such that lumen 122 of housing 110 is fluidly attached to lumen 203 of inflow conduit 205. Additionally, the length of collar 260 can be longer than inflow p/ort 120 to provide external support of inflow conduit 205, such as to prevent kinking of inflow conduit 205. In some embodiments, a tool is used to rotate collar 260, such as a torque-limiting wrench such as tool 500 of FIG. 7.

Referring now to FIGS. 9A and 9B, side sectional and perspective views, respectively, of a fluid absorbing plug is illustrated, consistent with the present inventive concepts. Plug 600 can include an outer surface 601, such as a cylindrical outer surface configured to engage in cylindrical inner surface of an electrical connector, such as an inner surface of connector 440 of FIG. 5 described hereabove. Plug 600 can include an inner surface 602, such as a cylindrical inner surface configured to engage a cylindrical outer surface of an electrical connector, such as a cylindrical outer surface of connector 140 of FIG. 6 described hereabove. Plug 600 can be comprises of fluid absorbing materials, such that plug 600 absorbs moisture surrounding an electrical connector and/or prevents fluids from adversely affecting an electrical connector. In some embodiments, plug 600 comprises a material selected from the group consisting of: foam; porous material; polyethylene; polyethylene foam; high-density polyethylene; ultra-high-molecular weight polyethylene; polytetrafluoroethylene; porous polytetrafluoroethylene; hydrophilic material; and combinations of these.

While the preferred embodiments of the devices and methods have been described in reference to the environment in which they were developed, they are merely illustrative of the principles of the inventions. Modification or combinations of the above-described assemblies, other embodiments, configurations, and methods for carrying out the invention, and variations of aspects of the invention that are obvious to those of skill in the art are intended to be within the scope of the claims. In addition, where this application has listed the steps of a method or procedure in a specific order, it may be possible, or even expedient in certain circumstances, to change the order in which some steps are performed, and it is intended that the particular steps of the method or procedure claim set forth herebelow not be construed as being order-specific unless such order specificity is expressly stated in the claim.

We claim:

1. A system for pumping blood in a patient comprising:
a rotational drive assembly;
a fluid drive element;
a housing, surrounding the fluid drive element, and comprising an inflow port and an outflow port;
an inflow cannula assembly comprising:
an inflow conduit with a proximal end and a distal end, wherein the distal end is operably attachable to the inflow port and the proximal end is fluidly connectable to a source of oxygenated blood; and
an inflow connector constructed and arranged to operably connect the inflow conduit to the inflow port; and
an outflow graft assembly comprising
an outflow conduit with a proximal end and a distal end, wherein the proximal end is operably attachable to the outflow port and the distal end is fluidly connectable to a blood vessel; and
an outflow connector constructed and arranged to operably connect the outflow conduit to the outflow port.

2. The system of claim 1 wherein the inflow cannula assembly is constructed and arranged to provide feedback of proper attachment.

3. The system of claim 2 wherein the feedback comprises feedback selected from the group consisting of: audible feedback; tactile feedback; force feedback; and combinations thereof.

4. The system of claim 1 wherein the inflow conduit comprises a first segment proximate the distal end and a second segment continuous with the first segment, wherein the second segment comprises a wall thickness and the first segment comprises a wall thickness thicker than the second segment wall thickness.

5. The system of claim 1 wherein the inflow conduit comprises a first segment proximate the distal end and a second segment continuous with the first segment, wherein the second segment comprises a reinforcing element along its length and the first segment does not include a reinforcing element.

6. The system of claim 5 wherein the inflow connector is constructed and arranged to be positioned on the inflow conduit first segment.

7. The system of claim 1 wherein the inflow connector is constructed and arranged to rotatably engage the inflow port.

8. The system of claim 1 wherein the inflow connector comprises at least one radially inward facing projection.

9. The system of claim 8 wherein the inlet port comprises a recess constructed and arranged to slidingly receive the at least one projection.

10. The system of claim 9 wherein the recess comprises a retention portion constructed and arranged to engage the at least one projection and prevent rotation of the inlet connector.

11. The system of claim 1 wherein the inflow connector comprises a bayonet locking element.

12. The system of claim 1 wherein the outflow graft assembly is constructed and arranged to provide feedback of proper attachment.

13. The system of claim 12 wherein the feedback comprises feedback selected from the group consisting of: audible; tactile; and combinations thereof.

14. The system of claim 1 wherein the outflow conduit comprises an inner layer, an outer layer and a hydrostatic barrier middle layer.

15. The system of claim 1 wherein the outflow connector is constructed and arranged to rotatably engage the outflow port.

16. The system of claim 1 wherein the outflow connector is constructed and arranged to be tightened to attach the outflow conduit to the outflow port, and wherein the outflow connector is further constructed and arranged to allow rotation of the outflow conduit at least one of during or after the tightening of the outflow connector.

17. The system of claim 16 wherein the outflow connector is further constructed and arranged to allow low-torque rotation of the outflow conduit at least one of during or after the tightening of the outflow connector.
